(19) United States
(12) Patent Application Publication
(10) Pub. No.: US 2020/0222306 A1
(43) Pub. Date: Jul. 16, 2020

(54) PERMANENT COLOURING COMPOSITION IN GEL OR CREAM FORM

(71) Applicant: LABORATOIRE ISATIS, LA ROCHELLE (FR)

(72) Inventors: Fabienne RAYNIER, PERIGNY (FR); Cécile GOUZON, LA ROCHELLE (FR)

(21) Appl. No.: 16/639,035

(22) PCT Filed: Aug. 17, 2018
(86) PCT No.: PCT/EP2018/072312

§ 371 Date: Feb. 13, 2020

(30) Foreign Application Priority Data
Aug. 17, 2017 (FR) FR 1770861

(51) Int. Cl. A61K 8/9789; A61Q 5/10; A61K 8/04

(52) U.S. Cl. CPC A61K 8/9789; A61K 2800/4324; A61K 8/042; A61Q 5/10

(57) ABSTRACT

The object of the invention is a composition in the form of a gel or cream comprising at least one solvent and at least one Lawsonia inermis extract, with this extract being present between 1 and 10% by weight of the total weight of the composition.

The subject of the invention is also a process for the manufacture of such a composition and its use in particular a process for permanent coloring of the hair and/or body hair.

### CROSS-REFERENCE TO RELATED APPLICATIONS

This application is the U.S. national phase application corresponding to PCT/EP2018/072312 which was assigned the international filing date of Aug. 17, 2018 and associated with publication WO 2019/034770 and which claims priority to French Patent Application FR 1770861 filed Aug. 17, 2017.

### TECHNICAL FIELD

This invention relates to the coloring of hair and body hair, in particular compositions based on natural coloring raw materials. In particular, the invention relates to compositions as gel or cream comprising an extract which enables the coloring to adhere to the hair fiber. The invention also relates to a process for obtaining such a composition and to its use for the permanent coloring of hair and/or body hair.

### BACKGROUND

Hair coloring has been around for a long time and is constantly evolving. Currently, a person who colors their hair has the choice between three ranges of product:
“oxidation” chemical colors, which represent the main class of product currently used. These products are composed of colorless precursors of dyes, called oxidation bases which, associated with strongly oxidizing products (hydrogen peroxide) and at a high pH, give rise, through a process of oxidative condensation, to colored compounds within hair fiber.
vegetable coloring consisting of a mixture of crushed plants applied as a poultice to the hair after dispersion in hot water. Three main plants are used for all the references currently sold: henna leaves (Lawsonia inermis: orange), indigo leaves (Indigofera tinctoria or gludolosa: blue) and cassia leaves (Cassia obovata: little coloring).
“Mixed” colors which are oxidation colors to which vegetable coloring extracts have been added. These compositions are formulated on the same principle as chemical colorings, with the addition of plant coloring extracts being more of a marketing argument than a real benefit in terms of shade obtained and safety.

Chemical or mixed coloring products, depending on their composition, are intended for different coloring modes:
Temporary colors: these are generally used to temporarily mask gray hair or to add a shade to the natural shade. The temporary coloring acts on the outside of the cuticle and adds a color to the surface of the hair. It cannot lighten dark hair and only very rarely withstands shampooing. They exist in several galenics: rinses, coloring foams, hair mascaras and coloring sprays, but also shampoos or colored care which “ensure” a relative long lifetime for the oxidation coloring.
Semi-permanent and semi-permanent coloring: these two coloring modes work in the same way, but the compositions differ in their formulation.
   Semi-permanent colors are designed to deposit color on the surface of the hair and have no lightening effect. The formula includes an activator that develops color and helps dilate the cuticle so that the coloring can settle there. The semi-permanent coloring does not penetrate the cortex and lasts only six to eight weeks, fading with the shampoo.
   The semi-permanent coloring is also deposited without lightening effect. The molecules used are smaller and penetrate the cortex of the hair, which allows them to be deposited on and in the cuticle. The semi-permanent color is said to be more effective in covering gray hair. The coloring lasts longer and does not fade with the shampoo.
Permanent oxidation dyes: these are made to durably penetrate the hair and deposit the color directly in the cortex of the hair. Their formulas contain a developer which dilates the cuticle and enables molecules to enter the cortex. These colorings also contain aniline derivatives which combine with hydrogen peroxide to produce larger tinted molecules which remain “trapped” in the cortex. Thanks to their formulation, permanent colors are capable of depositing colors, but they can also be used to lighten the shade of hair; all of this depends on the dose of hydrogen peroxide included in the formulation.

All these chemical or mixed coloring compositions are effective in terms of coloring, with a short exposure time (30 minutes) but have many drawbacks. In particular, these compositions generally consist of two products, a coloring base and an oxidant which must be mixed during application, which is not practical to use. In addition, they non-environmentally friendly and potentially toxic ingredients (dyes and additives), and require the use of irritants and/or allergenic products for both the have user and the applicator. In addition, these products lead to unsightly reflections over time and lead to the degradation of the hair fiber as the colorings progress. These products are therefore not satisfactory.

In particular, many people turn to natural hair coloring methods to eliminate the use of chemical constituents. Unlike chemical coloring compositions, they do not include toxic, allergenic or suspected carcinogenic ingredients such as PPD, Toluenes, Diamines, PEG, Aminophenol, Resorcinol and so on.

Vegetable coloring is historically recognized in particular in oriental beauty rites. Natural coloring coats the hair and forms a protective sheath around it. The coloring molecules attach to the upper layers of the hair fiber and gradually fade. The existing ranges of plant colors are composed mainly of powdered coloring plants, micronized soy proteins, alginate, thermal rock crystals, etc. In particular, some historic plants are known and currently used for, in particular, their ability to grip the hair. There are two classes of dye:
Henna which enables lasting grip on the hair which does not fade over time. But it has the major drawback of offering only warm, coppery tones. It provides a definite advantage in different types of formulation but deprives hairdressers and users of another category of colors, in particular cold tones such as dark chestnuts, neutral chestnuts and ashes.
Indigo which allows dark shades, but which adds unsightly green or even purple highlights. When it is applied, if the colorimetry work has not been optimized, indigo takes up considerable space on the hair and provides a very average and random end result.

These two plants also present supply problems and can be combined with metal salts to improve the coverage which breaks the hair.

In addition, natural colors are applied in one or two stages with a long exposure time (minimum 1.5 hours) and using a steam helmet which represents heavy investment and additional energy expenditure. The ranges are limited, the galenic poultice is crumbly and heavy (about 300 g of poultice to be applied consisting of 150 g of coloring powder which must be mixed with water) and it is unpleasant on application and rinsing both for the hairdresser and for the person having their hair colored. The color reproducibility is random, dark brown and blonde are very difficult to obtain, the colors degrade over time and grey hair coverage is insufficient.

### SUMMARY

The objective of the invention is to provide a product which overcomes all the drawbacks of the coloring products of the prior art, in particular a product which has the advantages of both natural colors and chemical colors, without presenting the disadvantages. To this end, the subject of the invention is a composition in the form of a gel or cream comprising at least one solvent and at least one extract of Lawsonia inermis, said extract being present between 1 and 10% by weight of the total weight of the composition.

Lawsonia inermis is a plant that grows naturally in the tropical and subtropical regions of Africa, South Asia and Australasia. It contains a particular, coloring molecule, lawsone (2-hydroxy-1,4-naphthoquinone) of formula:

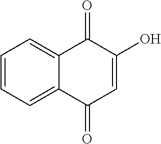 [SMILES: O=C1C=C(O)C(=O)c2ccccc21]

It also includes naphthoquinone derivatives, phenolic compounds, terpenoids, sterols, aliphatic derivatives, xanthones, coumarin, fatty acids, and amino acids.

Lawsonia inermis leaf powder has produced red, yellow and orange hues, which have been used in textile and body dyeing (hair coloring and care, temporary skin tattoos) for centuries. Its orange coloring is often very unsightly in certain formulations.

The composition according to the invention comprises an extract of Lawsonia inermis, mixed with a solvent, and in small quantities (less than 10%). Advantageously, the presence of this extract under these conditions enables:
if it is used alone in the composition to color the hair or the hairs blonde, and
if it is used in combination with one or more other coloring molecules, it makes it possible to improve the grip of the other coloring molecules which are fixed on it. Indeed, it advantageously provides the mordant dyeing necessary to accommodate all types of dye and does not color the hair fibers with the orange color of Lawsonia inermis when it is used in its natural state.

The composition according to the invention has many other advantages. It is a permanent color that works perfectly without synthetic products, is non-toxic and does not alter the hair fiber, with no unsightly highlights over time. In addition, the invention has a fast exposure time, a wide range of colors, color reproducibility, a pleasant and easy to use dosage form, there is no mixing to be carried out and the composition in gel or cream form is ready to use.

The invention also relates to a process for the manufacture of such a composition, and to its use for the permanent coloring of the hair.

### BRIEF DESCRIPTION OF THE DRAWINGS

FIG. 1 represents a scanning electron microscope image of a hair fiber damaged by chemical composition-type oxidation dyeing,

FIG. 2 represents a scanning electron microscope image of a hair fiber plant-colored by a plant coloration of the composition which is the subject of the invention,

FIG. 3 represents an image under an optical microscope of a hair fiber plant-colored by a plant coloration of the composition which is the subject of the invention.

### DETAILED DESCRIPTION OF THE INVENTION

Under the invention, “permanent coloring” means a lasting coloring over time, where after 3 weeks (conventional time between 2 colorings), a demarcation is visible between the previously-colored hair and the hair which has newly grown at the roots. This is called a “root effect”.

Under the invention “extract” of a raw material X means a set of molecules obtained from all or part of said raw material X, but excludes biomass, that is to say raw material or in powder form.

The subject of the invention is therefore a composition, in the form of a gel or a cream, comprising at least one solvent and at least one extract of Lawsonia inermis, with said extract of Lawsonia inermis being present between 1 and 10% by weight of the total weight of the composition.

The composition is therefore in the form of a cream or gel. Preferably, it has a viscosity (measured according to the Brookfield DV-I Prime method) of at least 400 mPa·s measured at 20° C.

It includes at least:
an extract of Lawsonia inermis,
a solvent.

The Lawsonia inermis extract present in the composition represents in total between 1 and 10% by weight of the total weight of the composition. Preferably, it represents at least 1.8% and/or at most 5% by weight of the total weight of the composition.

The Lawsonia inermis extract includes at least Loawsone. The rate of Lawsone present in this extract is between 0.05% and 2%. It may contain other molecules such as naphthoquinonic derivatives, phenolic compounds, terpenoids, sterols, aliphatic derivatives, xanthones, coumarin, fatty acids, and amino acids.

The extract is preferably an extract which has been enriched in amino acids. This amino acids in addition to those that may be present in the extract may have been added during or after extraction. The amino acids of the extract including those initially present as a result of the extraction and those optionally added, may represent in particular between 0.01% and 5% of the extract by weight of dry matter of the extract.

The extraction process is carried out from dry leaves of Lawsonia inermis, between 15 and 55° C., by solubilization, then comprises centrifugation, filtration, concentration and drying stages.

According to a particularly suitable production method, the Lawsonia inermis extract present in the composition according to the invention is an extract obtained from Lawsonia inermis leaves.

The Lawsonia inermis extract is mixed with a solvent in the cream or gel. The solvent preferably represents between 50 and 95% by weight of the total weight of the composition. The solvent present in the composition is a solvent which is compatible with cosmetic and/or hair applications. It is very preferably a solvent without a synthetic molecule and/or is non-toxic, and environmentally friendly. In particular, the solvent may be water or a hydro-alcoholic solution, and in particular a hydro-alcoholic solution comprising at least 50% water by weight or by volume.

When the Lawsonia inermis extract is the only coloring molecule present in the composition according to the invention, the coloration obtained when the composition is applied to white hair may be a blonde color, which is more or less pronounced depending on the amount of extract in the composition. When the Lawsonia inermis extract is the only coloring molecule present in the composition according to the invention, which is preferably in the form of a gel. In order to be able to obtain other colors, the composition according to the invention preferably comprises at least one other coloring molecule, in addition to the molecule(s) contained in the Lawsonia inermis extract. When the composition according to the invention preferably comprises at least one other coloring molecule, in addition to the molecule(s) contained in the Lawsonia inermis extract, this may be in the form of a gel or cream. The coloring molecule may be any coloring molecule, but it is preferably a coloring molecule of natural origin.

This coloring molecule may be used alone in the composition. Preferably, this coloring molecule is contained in an extract of raw material of natural origin and/or in a powder of a biomass of natural raw material. Therefore, the composition according to the invention may comprise, in addition to the Lawsonia inermis extract, at least one other extract of raw material of natural origin comprising at least one coloring molecule and/or at least one powder of a biomass natural raw material comprising at least one coloring molecule.

When the composition according to the invention comprises at least one extract of raw material of natural origin comprising at least one coloring molecule, each extract of raw material of natural origin in addition to the extract of Lawsonia inermis is preferably present between 0, 1 and 10% by weight of the total weight of the composition, and in particular between 0.5 and 7%.

When the composition according to the invention comprises at least one biomass powder of raw material of natural origin, each biomass powder of raw material of natural origin comprising at least one coloring molecule is preferably present between 0.5 and 50% by weight of the total weight of the composition, and in particular between 1 and 30%.

The raw material of natural origin of the extracts and/or powders comprising at least one coloring molecule present in the compositions according to the invention are preferably chosen from plants and/or shells and/or insects and/or microorganisms and/or lichens and/or fungi comprising at least one coloring molecule. They may, for example, be dye plants, and in particular:
plants containing quinones (present in madder, safflower, orcanette and so on) which form red, purple and red hues
anthraquinone rubiacs that provide reds
plants containing flavonoids that form yellow dye pigments
plants containing flavonoids which do not dye yellow: anthocyanins are pigments dissolved in the cellular juice of plants and chemical structures very similar to those of flavonoids, sources of yellow dyes. Most of the red, purple and blue hues of flowers and fruits around the world are due to these pigments.
indigo plants: dark blue coloring matter, differs from all other vegetable dyes: it does not exist directly in the many “indigo plants”. The indican and isatans A and B present in indigo plants are broken down by hydrolysis into colorless indoxyl and sugar. By grouping together 2 indoxyl molecules, indigo is formed in the presence of oxygen from the air.
tannin plants: that is to say substances of vegetable origin of astringent flavor. They are very widespread in the plant kingdom and certain botanical families are particularly rich in them: conifers, legumes, cashew nuts and fagaceae.

In particular, it may be one of the following raw materials of natural origin: Amla, Redwood, Buckthorn, Cachou, Logwood, Chestnut, Coreopsis, Eucalyptus, Garance, Broom, indigo, Mimosa, Mulberry dyers, Myrobalan, Nerprun, Dyers' pastel, Quebracho colorado, Reseda, Rosemary, Sappan, Solidage, Sorghum, Tara, Annatto, Walnut, Cinnamon, Turmeric, Henna, Hibiscus, Goldenseal, Katam, Ternate Liana, Oregon, Manjishta, Multani, Orcanette,

Rhapontic.

By way of example, the composition according to the invention can contain at least one of the extracts or powders presented in Table 1 below:

TABLE 1
examples of extracts and powders containing at least one coloring
molecule which may be present in the composition according to
the invention in addition to the extract of Lawsonia inermis

| INCI | Common name |
|---|---|
| Emblica officinalis extract | Amla extract |
| Haematoxylum brasiletto extract | Redwood extract |
| Frangula alnus bark extract | Buckthorn extract |
| Acacia catechu wood extract | Catechu extract |
| Haematoxylum campechianum wood extract | Logwood extract |
| Castanea sativa (chestnut) bark extract | Chestnut extract |
| Coreopsis tinctoria flower/leaf/stem extract | Coreopsis extract |
| Eucalyptus vegetable extract | Eucalyptus extract |
| Schinopsis balansae lorentzii | Brown extract |
| wood extract - sodium bisulphite | |
| Rubia tinctorum root extract | Standard madder extract |
| Genista tinctoria flower/leaf/stem extract | Broom extract |
| Indigofera tinctoria leaf extract | Indigo extract |
| Indigowoad leaf extract | Indigo leaf extract |
| Acacia mearnsii extract | Mimosa extract |
| Morus Tinctoria or Maclura Tinctoria extract | Dyers' mulberry extract |
| Terminalia chebula extract | Myrobalan extract |
| Rhamnus infectoria | Buckthorn extract |
| Isatis tinctoria extract | Dyers' Pastel Pigment |
| Schinopsis balansae wood extract | Quebracho extract |
| Schinopsis lorentzii Engl. | Quebracho extract |
| Reseda luteola flower/leaf/stem extract | Reseda extract |
| Rosmarinus officinalis extract | Rosemary extract |
| Caesalpinia sappan L. Extract | Sappan extract |
| Solidago canadensis flower/leaf/stem extract | Goldenrod extract |
| Sorghum bicolor leaf/stem extract | Sorghum extract |
| Caesalpinia paraguariensis extract | Tara extract |
| Amla fruit powder/Emblica officinalis | Amla powder |
| fruit powder | |
| Bixa orellana L. | Annato powder |
| Walnut stain powder | Walnut skin |
| Haematoxylum campechianum | Logwood |
| Cinnamomum zeylanicum powder | Cinnamon |
| Turmeric longa root powder | Turmeric |
| Rubia tinctorum root powder | Madder powder roots |
| Lawsonia inermis | Henna powder |
| Cassia obovata | Neutral henna |
| Hibiscus sabdariffa flower powder | Hibiscus rosa |
| | flower powder |
| Hydrastis Canadensis | Goldenseal root powder |
| Indigofera tinctoria leaf powder | Indigo leaf powder |
| Buxus dioica leaf powder | Katam |
| Clitoria ternatea | Ternate liana |
| Berberis acquifolium | Oregon Grape Root |
| | powder |
| Rubia cordifolia root powder (Indian madder) | Manjishta Powder |
| Solum fullonum | Multani Mithi |
| Alkanna tinctoria root powder | Orcanette powder |
| Rhapontic root powder | Rhapontic |

In addition to the coloring molecules and the solvent, the composition according to the invention may contain one or more excipient(s), that is to say any component other than the coloring molecules (or the extracts or powders containing them) and the solvent. Preferably, these excipients are non-toxic products for humans and the environment and can be of natural origin.

These other components are preferably chosen from:
preservatives, such as for example those of natural origin such as naticide, grapefruit extract, rosemary, vitamin E, propolis, etc. or those of synthetic origin that can be labeled organic, such as benzyl alcohol, dehydroacetic acid, etc.
Texturing agents, in particular texturing agents which make it possible to obtain a texture in the form of a cream or gel, such as, for example, guar, acacia, xanthan, arabic gums, etc.
Perfume adjusters, such as, for example, denaturing products and odor adjusters,
Perfume agents
PH adjusters, such as for example soda, citric acid, lactic acid, etc.
hair and/or cosmetic active ingredients, in particular active ingredients capable of improving the final quality of the hair, such as, for example, phytokeratin, spirulina, glycerin, etc.
oils, especially vegetable oils.
essential oils, in particular for their perfuming and antioxidant properties, such as for example the essential oils of rosemary, turmeric, grapefruit, cloves, etc.
surfactants, natural or synthetic, which may be labeled organic.
dye powders, that is to say mixtures of plant powders, plant extracts, pH adjusters or any other natural material that make it possible to intensify the final color rendering.

These excipients, when present, each preferably represent between 0.05 and 10% by weight of the composition.

The composition according to the invention can be obtained by the implementation of any suitable process. Preferably, it is obtained by implementing a manufacturing process that comprises the following steps:
Mix the Lawsonia inermis extract with the solvent, stirring at a temperature between 15 and 80° C. for 5 minutes to two hours,
Optionally add one or more other coloring molecule(s) and/or one or more raw material extract(s) of natural origin and/or one or more biomass powder(s) of raw material of natural origin containing one or more other coloring molecule(s) and mix, stirring at a temperature between 15 and 80° C. for 5 minutes to two hours,
Add any excipient(s) and mix, stirring at a temperature between 15 and 80° C. for 5 minutes to two hours.

The mixing speed is preferably at least 50 revolutions/minute, preferably at least 1000 revolutions/minute. It may in particular be between 50 and 3000 revolutions/minute, preferably between 1000 and 3000 revolutions/minute.

According to a variant, the temperature at which each mixing is carried out is between 40° and 70° C. In this case, each mixing operation lasts preferably between 5 and 30 minutes.

According to another variant, the temperature at which each mixing is carried out is ambient temperature (in particular between 15 and 30° C.). In this case, each mixing operation lasts preferably between 30 minutes and 2 hours.

The composition according to the invention can preferably be stored for at least 30 months. It can be packaged in any packaging that enables creams or gels to be stored and conserved, in particular any opaque packaging (which protects the dyes from daylight), stable (without migration effect of the product towards the packaging and vice versa), such as PE/Aluminum/PET triplex packaging. Advantageously, the compositions according to the invention, when applied to the hair or the hairs, make it possible to color them durably, with a beautiful color, without unsightly highlights and very good resistance over time, while being able to be constituted mainly or only of natural origin, non-toxic and non-irritant components. The invention makes it possible to propose a wide range of colors for the permanent coloring of the hair with a rapid exposure time, of 1.5 hours maximum, and generally less than or equal to one hour.

The invention therefore also relates to the use of the composition according to the invention for the permanent dyeing of hair or body hair.

In particular, the invention relates to a process for permanent coloring of the hair, comprising the implementation of the following steps:
apply the composition according to the invention to all of the clean hair,
leave on for 10 minutes to 1 hour 30 minutes,
rinse the hair, preferably with clear water.

The hair must be clean before applying the coloring composition according to the invention. Therefore, the hair is preferably washed and rinsed before the composition is applied. It may, however, be dry or wet.

The composition may, for example, be applied with a brush by spreading it in lines from the front of the hair to the back. A hair net or food-type film is then preferably placed on the hair. For at least part of the composition's exposure time, the hair may be subjected to heat, in particular between 35 and 65° C., to enable the exposure time to be sped up and encourage the attachment of the coloring molecules in particular. For example, a heated cap may be used.

Finally, the hair may potentially be washed after the exposure time before being rinsed. The invention is now illustrated by examples and by results of tests on the quality of the hair after coloring according to the invention in comparison with a permanent coloring of the prior art.

### EXAMPLES

Table 2 below provides the constitution of several composition examples according to the invention.

| | Raw | Percentage |
|---|---|---|
| Color | material | by weight |
| Blond | Demineralized Water | 81.00% |
| | Vegetable Ethyl Alcohol | 10.00% |
| | Lowsonia inermis extract | 3.00% |
| | Xanthan gum | 5.00% |
| | Geogard221 | 1.00% |
| Copper Red | Demineralized Water | 69.80% |
| | Henna powder | 10.00% |
| | Sorghum extract | 8.00% |
| | Lawsonia inermis extract | 5.00% |
| | Vitamin E | 5.00% |
| | Phytokeratine | 2.00% |
| | Fragrance | 0.10% |
| | Citric acid | 0.10% |
| Brown chestnut | Demineralized Water | 81.00% |
| | Sodium hydroxide | 5.00% |
| | Annato powder | 5.00% |
| | Logwood extract | 4.00% |
| | Lawsonia inermis extract | 2.00% |
| | Ecogel | 2.00% |
| | Naticide | 1.00% |

These compositions may be obtained as follows:
first solubilization:
   weigh the extract of Lawsonia inermis and place it in tank 1,
   add cosmetic grade water,
   mix, stirring at at least 1000 revolutions/minute at 40° C. for 30 minutes,
   add a pH adjuster to tank 1,
   mix, stirring at least 1000 revolutions/minute, heat to 40° C. for 30 minutes and store in this condition in tank 1,
add the other components (step to be repeated for each component or to be done at once for several components):
   weigh the component to be added,
   add to tank 1,
   mix, stirring at at least 1000 revolutions/minute, heat to 40° C. for 30 minutes and store in this condition in tank 1.

### Tests

The compositions presented in the examples were tested on locks of hair according to the protocol described below, and compared with coloring of the same locks with an oxidation coloring product of chemical type composition of the prior art of the following formula:

| Ingredient | Percentage by weight |
|---|---|
| Surfactants (ammonium laureth sulfate, | 60 |
| laureth-12) | |
| Emulsifiers (ethoxylated fatty alcohols) | 30 |
| Soaps (ammonium hydroxide/ethanolamine | 30 |
| oleates) | |
| Solvents (propylene glycol, benzyl alcohol) | 20 |
| Ethanol and/or isopropanol | 16 |
| Hair dyes | 12 |
| Alkaline agents | 10 |
| Viscosity agents (fatty alcohols, carbomer) | 10 |
| Cationic surfactants (dicetyldimonium | 5 |
| chloride, PEG-2, oleamine) | |
| Cationic polymers (polyquaternium-34) | 5 |
| Hair conditioners and pearlescent | 5 |
| Reducing agents (sodium metabisulfite) | 3 |
| Perfume | 2 |
| Sequestrant (EDTA) | 2 |
| Water | Qsp 100 |

The operating protocol is described below.
Wash a lock of hair weighing between 0.200 g and 0.500 g, with a neutral pH shampoo,
Apply between 1.5 g and 3 g of composition according to the invention to the lock, so that the lock of hair is covered in a generous and sufficient amount,
Place under film,
Leave under heat for 1 hour, rinse and dry, then observe the color result and compare with the control sample,
Leave to cool to room temperature,
Observe under a scanning electron microscope and an optical microscope after 48 hours.

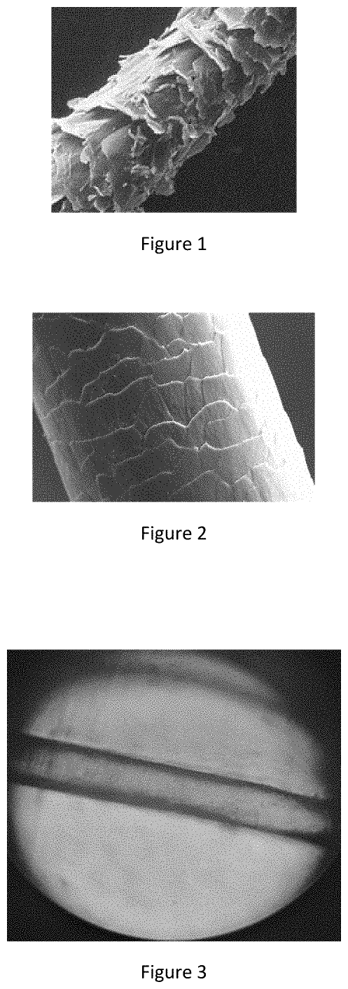

The results are presented in FIG. 1 for a lock treated according to the prior art and in FIGS. 2 and 3 for a lock treated with the product according to the invention. It is found that for hair colored with a composition according to the invention, the hair scales are perfectly smooth, unlike hair treated with a coloring of the prior art in which the scales are detached, with the hair structure being irreparably altered.

In addition, in FIG. 3, it can be seen that the composition according to the invention is deposited well on the hair fiber.

1. A composition for the permanent coloring of hair or body hairs in the form of a gel or cream comprising at least one solvent and at least one extract of Lawsonia inermis comprising Lawsone, with this extract being present between 1 and 10% by weight of the total weight of the composition.

2. The composition according to claim 1, characterized in that the solvent is chosen from water and an aqueous-alcoholic solution.

3. The composition according to claim 1, characterized in that the solvent represents between 50 and 95% by weight of the total weight of the composition.

4. The composition according to claim 1, characterized in that the extract of Lawsonia inermis is present in an amount of at least 1.8% and/or at most 5% by weight relative to the total weight of the composition.

5. The composition according to claim 1, characterized in that the Lawsonia inermis extract is an extract in which amino acids have been added.

6. The composition according to claim 1, characterized in that the amino acids represent between 0.01% and 5% of the extract by weight of dry matter of the extract.

7. The composition according to claim 1, characterized in that the Lawsonia inermis extract is an extract obtained from the leaves of Lawsonia inermis.

8. The composition according to claim 1, characterized in that it does not comprise any other coloring molecule in addition to the Lawsonia inermis extract and in that it is in the form of a gel.

9. The composition according to claim 1, characterized in that the rate of Lawsone present in the Lawsonia inermis extract is between 0.05% and 2%.

10. The composition according to claim 1, characterized in that it also comprises at least one other coloring molecule.

11. The composition according to claim 1, characterized in that said at least one other coloring molecule is of natural origin.

12. The composition according to claim 1, characterized in that it comprises, in addition to the Lawsonia inermis extract, at least one other extract of raw material of natural origin comprising at least one coloring molecule and/or at least one powder from a biomass of natural raw material comprising at least one coloring molecule.

13. The composition according to claim 1, characterized in that each other extract of raw material of natural origin is present between 0.1 and 10% by weight of the total weight of the composition.

14. The composition according to claim 12, characterized in that each biomass powder of raw material of natural origin is present between 0.5 and 50% by weight of the total weight of the composition.

15. The composition according to claim 12, characterized in that the raw material of natural origin is chosen from plants and/or shells and/or insects and/or microorganisms.

16. The composition according to claim 12, characterized in that the raw material of natural origin is chosen from the following raw materials of natural origin:
Amla, Redwood, Buckthorn, Cachou, Logwood, Chestnut, Coreopsis, Eucalyptus, Garance, Broom, indigo, Mimosa, Mulberry dyers, Myrobalan, Nerprun, Dyers' pastel, Quebracho colorado, Reseda, Rosemary, Sappan, Solidage, Sorghum, Tara, Annatto, Walnut, Cinnamon, Turmeric, Henna, Hibiscus, Goldenseal, Katam, Ternate Liana, Oregon, Manjishta, Multani, Orcanette, Rhapontic.

17. The composition according to claim 1, characterized in that it comprises at least one other component chosen from preservatives, texturing agents, pH adjusters, essential oils, oils, surfactants, dye powders, hair and/or cosmetic active ingredients, and perfume agents.

18. The composition according to claim 1, characterized in that it has a viscosity of at least 400 mPa·s.

19. A method for manufacturing the composition according to claim 1, characterized in that it comprises the following stages:
Mix the Lawsonia inermis extract with the solvent, stirring at a temperature between 15 and 80° C. for 5 minutes to two hours,
Optionally add one or more other coloring molecule(s) and/or one or more raw material extract(s) of natural origin and/or one or more biomass powder(s) of raw material of natural origin containing one or more other coloring molecule(s) and mix, stirring at a temperature between 15 and 80° C. for 5 minutes to two hours,
Add any excipient(s) and optionally a dye powder and mix, stirring at a temperature between 15 and 80° C. for 5 minutes to two hours.

20. Use of the composition according to claim 1, for the permanent coloring of hair or body hair.

21. A permanent hair coloring process, characterized in that it comprises the implementation of the following stages:
Apply the composition according to claim 1 to all of the clean hair,
Leave on for 10 minutes to 1 hour 30 minutes,
Rinse the hair.

22. The process for permanent coloring of hair according to claim 21, characterized by at least one of the following characteristics:
The hair is washed beforehand and rinsed before the coloring composition is applied,
The hair is subjected to heat for at least part of the time when the composition is applied to the hair,
The hair is washed after the exposure time before being rinsed.
